(19) United States
(12) Patent Application Publication
(10) Pub. No.: US 2014/0152443 A1
(43) Pub. Date: Jun. 5, 2014

(54) POSTURE TRAINING DEVICE HAVING MULTIPLE SENSITIVITY LEVELS AND BOTH POSITIVE AND NEGATIVE FEEDBACK

(71) Applicants: Garrett L. Cammans, Orem, UT (US); Lance J. Larson, Orem, UT (US); Jeremy C. Smith, Orem, UT (US)

(72) Inventors: Garrett L. Cammans, Orem, UT (US); Lance J. Larson, Orem, UT (US); Jeremy C. Smith, Orem, UT (US)

(21) Appl. No.: 13/964,023

(22) Filed: Aug. 9, 2013

Related U.S. Application Data
(60) Provisional application No. 61/681,346, filed on Aug. 9, 2012.

(51) Int. Cl. A61B 5/11

(52) U.S. Cl. CPC A61B 5/1116
USPC 340/573.7

(57) ABSTRACT

A posture training device is immovably attached to a user's chest. On the exterior of the device, there are three LEDs, an ON/OFF button and a selector button. When the ON/OFF button is first pressed, the device boots to the ON state. A sensitivity level, indicated by one of the LEDs, is selected by momentarily pressing the selector button one to three times. A user presses and holds the central selector button, while standing or sitting in a desired posture position until he feels a vibration. The device locks on to the selected position one second after selector button release. Vibrational negative feedback is provided whenever the user slumps or slouches outside the limits permitted by the chosen sensitivity level. Vibrational positive feedback is provided when correct posture is reestablished. When the ON/OFF button is pressed again, the device shuts OFF.

### BACKGROUND OF THE INVENTION

1. Field of the Invention

The present invention relates generally to electronic devices designed to be worn for the purpose of monitoring the wearer's posture and, more particularly, to such electronic devices which provide automatic feedback to the wearer.

2. History of the Prior Art

Good posture is generally regarded as the position in which the body is held upright in opposition to gravity while standing and sitting. Maintenance of good posture involves training the body to stand, walk and sit such that the least amount of strain is placed on supporting muscles and ligaments. Proper posture keeps bones and joints in the correct alignment so that muscles are being used properly, helps decrease the abnormal wearing of joint surfaces, decreases the stress on the ligaments holding the joints of the spine together, prevents the spine from becoming fixed in abnormal positions, minimizes fatigue because muscles are being used more efficiently, minimizes the likelihood of backache and muscular pain, and contributes to a good appearance. In order to have proper posture, one must have good muscle flexibility, a normal range of motion in the joints, strong postural muscles, a balance of muscles on both sides of the spine, and an awareness of one's own posture, plus awareness of proper posture which leads to conscious correction.

Good posture is essential not only for optimum health, but for success in most of life's endeavors. Posture is often the first characteristic about us that others notice, and that first impression can lead to assumptions about our personalities. Those with good posture are generally perceived as being energetic, confident and assertive, while those with bad posture may be considered as lacking confidence, being overly cautious, sad or even negative. Bad posture may discourage others from developing relationships with us, and will likely limit our success in business, social interactions, and relationships with the opposite sex. It this is not sufficient reason to maintain good posture, maybe it is worth noting that those with good posture enjoy far less back and neck pain and have far fewer long-term back injuries.

It has been established that a key element of truly good posture is the inclination of the head in the saggital plane (the median vertical plane dividing the body into right and left halves), along with a necessary linear elongation of the neck and spine. There is an optimal range of head inclination within which the weight of the head tends to be balanced and thus minimize the amount of force required from the supporting muscles. Generally, individuals whose normal head positioning is held within this optimal range tend to enjoy physical wellbeing and good appearance. Outside this optimal range, unbalancing of the weight of the head upsets the muscle equilibrium, and, if continued over a length of time, generally leads to symptoms of stress and discomfort with risk of progressive deterioration and deformity with aging, along with the resultant disadvantages to the appearance and self-esteem.

For those who seek to improve their posture, a training program with biofeedback and positive reinforcement will be of benefit. Conventional techniques rely heavily on the verbal admonishments of others, including professionals, and may require extraordinary high levels of self-discipline and self-awareness, which are possessed by very few individuals.

There is a need for a simple posture training device which can be worn under the person's outer clothing. The device must be able to accurately detect both correct and incorrect posture and provide a user with both negative and positive feedback with respect thereto. It should also have multiple sensitivity levels so that as a trainee becomes more proficient at maintaining proper posture, the sensitivity of the device can be elevated. Likewise, for an individual who is just beginning a training regimen, a device setting of reduced sensitivity will eliminate constant negative feedback that may result in discouragement. One aspect of proper posture involves bending the body at the waist, for example, in order to pick up objects located at lower than standing level. Though a person bending at the waist can employ either proper or improper posture, all available posture monitoring devices will signal the wearer of an improper posture condition. The key to proper posture while bending at the waist is to maintain the shoulders pulled back. Thus, maintaining optimum distance between the shoulders is the key to proper posture. A posture monitoring device should, therefore, take into account this aspect of posture.

### SUMMARY OF THE INVENTION

The applicant's posture training device is a disc-shaped device that can either be adhesively adhered directly to the chest beneath the collar bone or be secured to a cord looped around the neck. To ensure accuracy of the device, it is preferable to wear form-fitting clothing that will hold the posture training device against the user's chest, in order to reduce motion or movement which may occur with loose fitting clothing. The posture training device is simple to operate. On the exterior of the device, the user sees two momentary-contact switch buttons: an ON/OFF button and a central selector button. Also visible are three light emitting diodes (LEDs) 1, 2 and 3. When the ON/OFF button is pressed, LED 3 lights up, followed by LED 2 and, then, by LED1. Once all three LEDs are simultaneously lit up to indicate an ON state, power to the LEDs is minimize current drain on the 3-volt electrochemical cell that powers the unit. Once the device is in an ON state, the sensitivity level is selected by pressing the selector button momentarily one to three times. If pressed one time, LED 1 flashes once, which indicates that maximum sensitivity has been selected; if pressed a second time, LED 2 flashes once, which indicates that medium sensitivity has been selected; if pressed a third time, LED3 flashed once, which indicates that minimum sensitivity has been selected. In order to set the desired posture position setting, the user, while sitting or standing with his desired posture, presses and holds the central selector button until he feels a vibration. Upon feeling the vibration, the user releases the central button quickly and places his arms down at his side for one second, which allows the device to lock in the selected position. Negative feedback, consisting of a very quick, is provided to the user whenever he slumps or slouches outside the limits permitted by the chosen sensitivity level. Positive feedback, consisting of two very quick vibrations, are provided to the user when he returns to the selected posture position. When the ON/OFF button is pressed a second time, all three LEDs simultaneously light up. They then sequentially shut off, with LED 1 being the first to shut off, followed by LED2, and then LED3. Once LED 3 has shut off, the device is in an OFF state. When the device is turned ON again by pressing the ON/OFF button, the device defaults to the last used settings. Thus, there is no need to recalibrate the device for each use.

For enhanced posture monitoring capability, two interacting posture monitoring devices can be simultaneously worn, with one device attached to each shoulder. Each device incorporates a miniature radio transceiver and circuitry for accurately measuring the distance between devices using precision calculations based on both coarse resolution and fine resolution attributes. Such a measurement technique is disclosed in U.S. Pat. No. 6,922,166. During calibration of the devices, both inclination of each device and distance between the devices is recorded. Allowing the shoulders to sag will produce negative feedback from one or both devices. However, bending at the waist will produce negative feedback only when distance between the shoulders decreases beyond limits determined by sensitivity settings of the devices.

A presently preferred embodiment of the invention is packaged in a disc shaped case having a diameter of about 40.25 mm (1.584 inches) and a thickness of about 13.7 mm (0.538 inches). The case holds a generally circular, dual-layer printed circuit board. The front side of the circuit board is populated with three light emitting diodes (LEDs), a first micro switch that is actuated by the ON/OFF button, a second micro switch that is actuated by the central selector button, a small package One-Time-Programmable 46R01B 8-bit Micro Controller Unit (OTP MCU), a small, low-power 335B three-axis ±3 g accelerometer, a Schottkey diode, an A106 Voltage Controlled Oscillator (VCO), a 702 MOSFET, and various capacitors and resistors. The back side of the circuit board is populated with an electrochemical cell clip which holds a 3-volt CR2032 coin-shaped battery, and a micro electric DC motor having its output shaft coupled to an out-of-balance rotating weight. The motor and the rotating weight provide vibration pulses which are used for calibration, as well as for positive and negative feedback.

### BRIEF DESCRIPTION OF THE DRAWINGS

FIG. 1 is a top plan view of the assembled posture training device;

FIG. 2 is a bottom plan view of the assembled posture training device;

FIG. 3 is a bottom plan view of the posture training device with the battery cover removed and the internal battery exposed;

FIG. 4 is a top plan view of the circuit board of the posture training device;

FIG. 5 is a bottom plan view of the circuit board of the posture training device; and

FIG. 6 is a block diagram of a master enhanced posture training device having a transceiver and distance measuring capability integrated with the inclinometer and feedback circuitry;

FIG. 7 is a block diagram of a slave posture training device having a transceiver and distance measuring capability;

FIG. 8 is a side elevational view of a person having correct sitting posture;

FIG. 9 is a side elevational view of a person having incorrect, slouching sitting posture;

FIG. 10 is a side elevational view of a person having incorrect, leaning-forward sitting posture;

FIG. 11 is a side elevational view of a person having correct standing posture;

FIG. 12 is a side elevational view of a person having incorrect standing posture, with the head protruding forward;

FIG. 13 is a side elevational view of a person having incorrect standing posture, with rounded shoulders;

FIG. 14 is a side elevational view of a person having incorrect standing posture, with an arched back;

FIG. 15 is a side elevational view of a person having incorrect standing posture, with forward leaning head; and

FIG. 16 is a side elevational view of a person having incorrect, slumping standing posture.

### DETAILED DISCLOSURE OF THE INVENTION

The posture training device will now be described in detail with reference to the attached drawing figures. It should be understood that though the drawings are required to be merely representative of the invention, a reasonable attempt has been made to draw the device and the enclosed circuit board to scale.

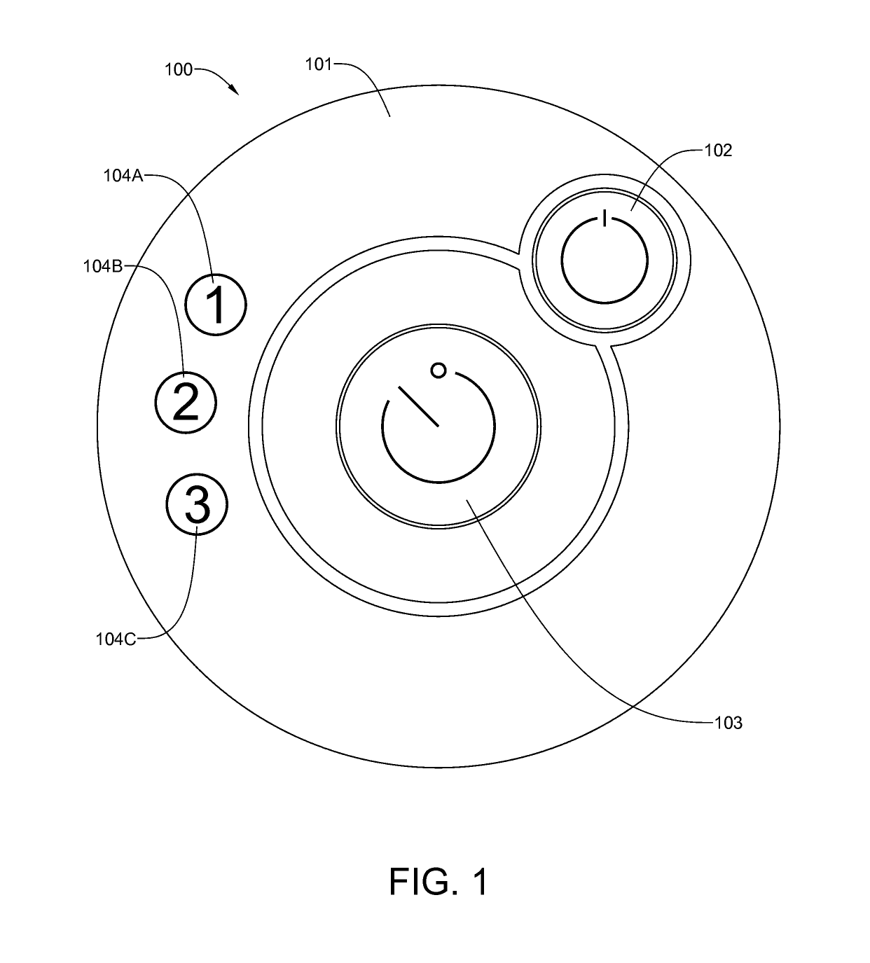

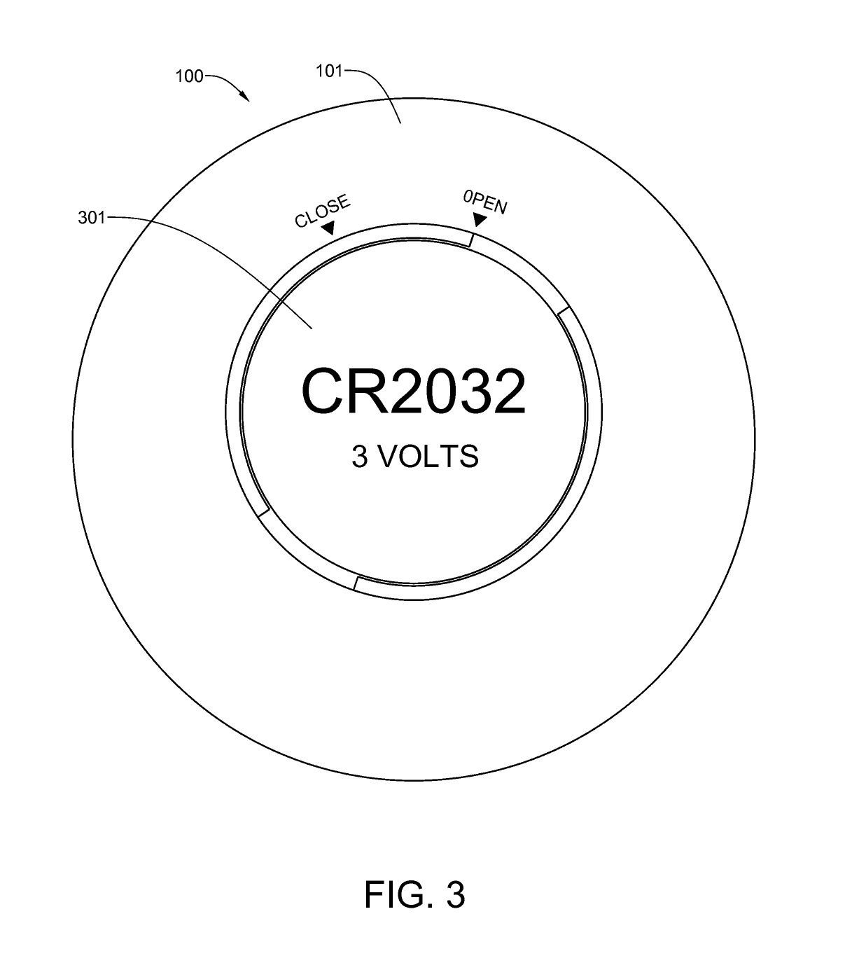

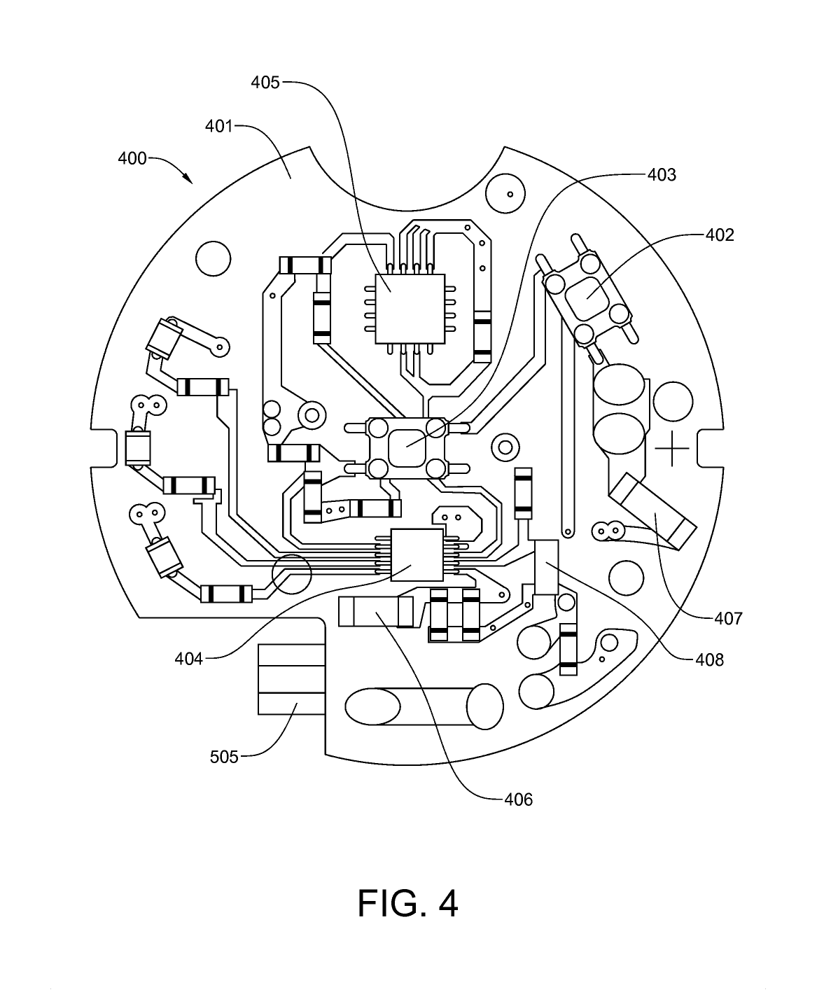

Referring now to FIG. 1, a presently preferred embodiment of the posture training device 100 is packaged in a disc shaped case 101 having a diameter of about 40.25 mm (1.584 inches) and a thickness of about 13.7 mm (0.538 inches). The case 100 holds a generally circular, dual-layer printed circuit board and a 3-volt battery, which will subsequently be described in detail with reference to drawing FIGS. 3 and 4. On the exterior front face of the case, the user sees two momentary-contact switch buttons: an ON/OFF button 102 and a central selector button 103. Also visible are three light emitting diodes (LEDs) 104A, 104B and 104C that are labeled 1, 2 and 3, respectively. When the ON/OFF button 102 is pressed, LED 3 (104C) lights up, followed by LED 2 (104B) and, then, by LED 1 (104A). Once all three LEDs are simultaneously lit up to indicate an ON state, power to the LEDs is shut off, thereby minimizing current drain on the 3-volt electrochemical cell that powers the unit. Once the posture training device 100 is in an ON state, an appropriate sensitivity level is selected by pressing the selector button 103 momentarily one to three times. If pressed one time, LED 1 (104A) flashes once, which indicates that maximum sensitivity has been selected; if pressed a second time, LED 2 (104B) flashes once, which indicates that medium sensitivity has been selected; if pressed a third time, LED3 (104C) flashes once, which indicates that minimum sensitivity has been selected. In order to set the desired posture position setting, the user, while sitting or standing with his desired posture, presses and holds the central selector button 103 until he feels a vibration. Upon feeling the vibration, the user releases the central button 103 quickly and places his arms down at his side for one second, which allows the device to lock in the selected position. Negative feedback, consisting of a very quick, is provided to the user whenever he slumps or slouches outside the limits permitted by the chosen sensitivity level. Positive feedback, consisting of two very quick vibrations, are provided to the user when he returns to the selected posture position. When the ON/OFF button 102 is pressed a second time, all three LEDs simultaneously light up. They then sequentially shut off, with LED 1 (104A) being the first to shut off, followed by LED2 (104B), and then LED3 (104C). Once LED 3 (104C) has shut off, the device is in an OFF state. When the device is turned ON again by pressing the ON/OFF button, the device defaults to the last used settings. Thus, there is no need to recalibrate the device for each use.

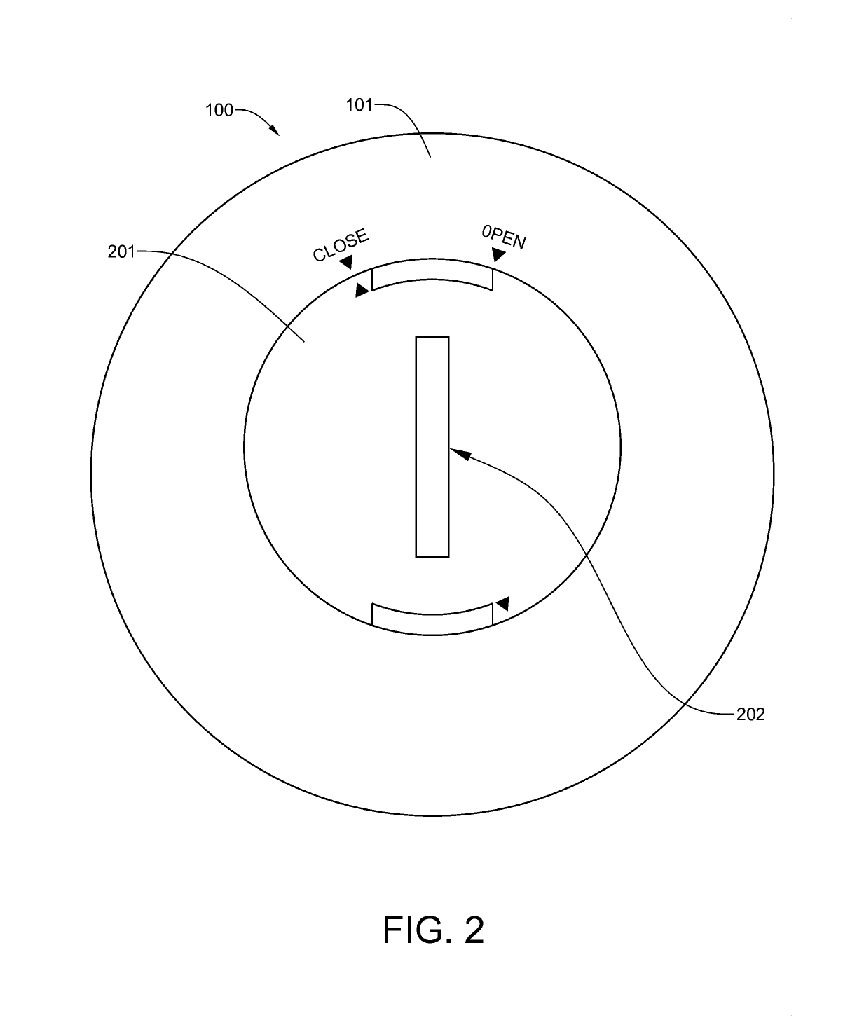

Referring now to FIG. 2, on the exterior rear face of the case, the user sees a circular battery cover 201 that can be rotated counterclockwise approximately 30 degrees, with a coin inserted in a coin slot 202, in order to disengage it from the case 101. The battery cover 201 will fall out when the device 100 is turned front face up. Removal of the battery cover 201 exposes a compartment that houses a 3-volt coin-shaped, two-cell battery. The battery cover 201 can be removed in order to replace the battery after it has completely discharged.

Referring now to FIG. 3, the battery cover has been rotated clockwise and removed, thereby exposing the internal 3-volt CR2032 coin-shaped battery 301.

Referring now to FIG. 4, the front side of the circuit board 400 is populated with three light emitting diodes (LEDs) 104A, 104B and 104C, a first micro switch 401 that is actuated by the ON/OFF button 102, a second micro switch 402 that is actuated by the central selector button 103, a small package One-Time-Programmable 46R01B 8-bit Micro Controller Unit (OTP MCU) 403, a small, low-power 435B three-axis ±3 g accelerometer 404, a Schottkey diode 405, an A106 Voltage Controlled Oscillator (VCO) 406, a 702 MOSFET 407, and various capacitors and resistors.

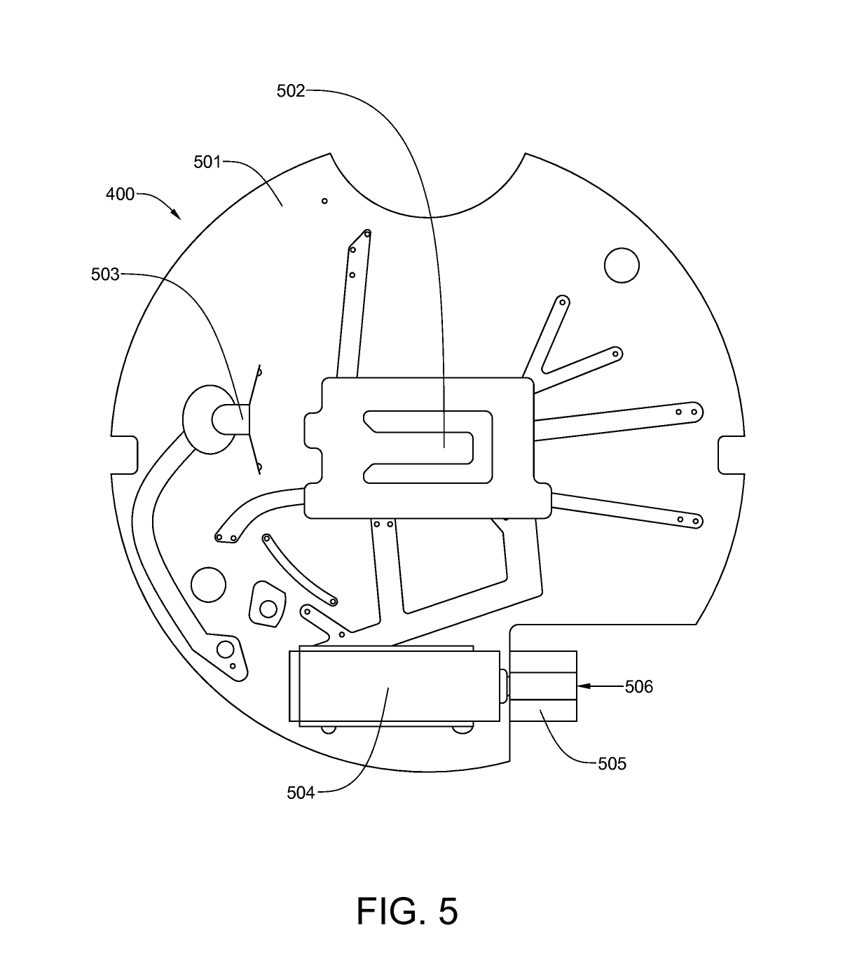

Referring now to FIG. 5, the back side 501 of the circuit board 400 is populated with a negative electrochemical battery contact 502 and a positive battery clip 503, which together make electrical contact with the CR2032 coin-shaped battery 301, a micro electric DC motor 504 having a rotatable, out-of-balance weight 505 coupled to the output shaft 506 of the motor 504. The motor 504 and the rotating weight 505 provide vibration pulses which are used for calibration, as well as for positive and negative feedback.

For enhanced posture monitoring capability, a master enhanced posture monitoring device and two slave devices can be simultaneously worn, with the master device attached to the chest and a slave device attached to each shoulder. Each of the three devices is equipped with a miniature radio transceiver and circuitry for accurately measuring the distance between the two devices, in addition to the circuitry disclosed in the descriptions of FIGS. 4 and 5, but also with a 3 and 4. Distance between the master device and each of the two slave devices is calculated as a function of the time required for signal transmission between the two devices. Such a measurement technique is disclosed in U.S. Pat. No. 8,274,426 (the '426 Patent), titled HIGH-RESOLUTION, ACTIVE REFLECTOR RADIO FREQUENCY RANGING SYSTEM, to Daniel Joseph Lee. The invention of the '426 Patent provides ultra compact circuitry capable of measuring distances within 0.125 mm. The '426 Patent is hereby incorporated by reference in the present application. It is envisioned that the invention of the '426 Patent can be incorporated into the enhanced master posture monitoring device 600 and into the slave devices 700 to effectuate accurate distance measuring. Because the approximate distance is known, absolute distance measurements can be calculated without frequency hopping. Distance may also be determined by another method in which one device transmits a signal having a known phase relationship at the point of transmission. Distance can be calculated from the phase relationship of the signal at the point of reception. Wavelength of the transmitted signal is selected so that the distance between the two points will never vary more than a wavelength of the transmitted signal. The frequency is determined experimentally on a range of subjects wearing a device on each shoulder. The highest level of measurement accuracy is obtained when the difference between minimum and maximum distances is close to a full wavelength. During calibration of the devices, both inclination of each device and distance between the devices is recorded by the devices. Allowing the shoulders to sag will produce negative feedback from one or both devices. However, bending at the waist will produce negative feedback only when distance between the shoulders decreases beyond limits determined by sensitivity settings of the devices.

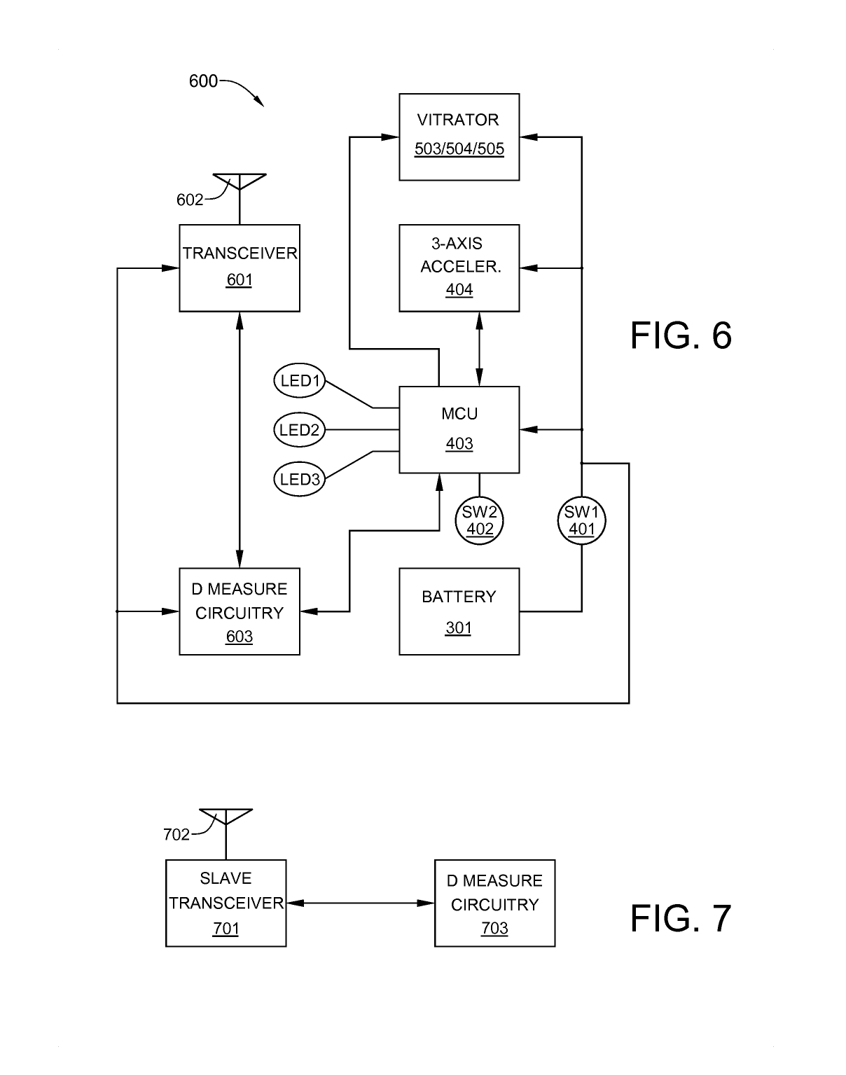

Referring now to FIG. 6, an enhanced master posture monitoring device 600 includes not only the inclination monitoring circuitry disclosed in FIGS. 4 and 5, but also a transceiver 601 having a transmit-receive antenna 602 and distance measuring circuitry 603, which precisely measures the distance between it and two slave devices 600 which may be rigidly affixed to each shoulder of a user.

Referring now to FIG. 7, a slave device 700 having a slave transceiver 701 and measurement circuitry 702 does not include the posture measuring circuitry of the master device 600. The only function of the slave device is to provide accurate measurements between it and the enhance posture monitoring device 600. As long as the distance between the enhance posture monitoring device 600 and either of two slave devices 700 does not change within a set range when the user, for example, bends at the waist using proper posture, the vibrator 503/504/505 will not be activated. However, the vibrator will activate to alert to any posture degradation, whether the user is erect or inclined at the waist.

Referring now to FIG. 8, a person sitting with correct posture has ear, shoulder and hip aligned in a straight, vertical line. Proper posture is characterized by having the head straight, shoulders back, chest out, stomach in, back supported and legs supported. The posture training device 100 is designed to maintain this correct sitting posture by signaling to a user when correct posture is forsaken, and also signaling to the user when correct posture is resumed.

Referring now to FIG. 9, a person sitting with incorrect slouching posture has the head protruding forward, body tilted back, shoulders rounded, upper back rounded, and lower back rounded.

Referring now to FIG. 10, a person sitting with incorrect leaning-forward posture has the head forward, shoulders rounded forward, chest leaning forward, and back arched forward.

Referring now to FIG. 11, a person standing with correct posture has ear, shoulder, hip, and ankle in a straight vertical line. The posture training device 100 is designed to maintain this correct standing posture by signaling to a user when correct posture is forsaken, and also signaling to the user when correct posture is resumed.

Referring now to FIG. 12, a person standing with incorrect head-forward standing posture has the head protruding forward.

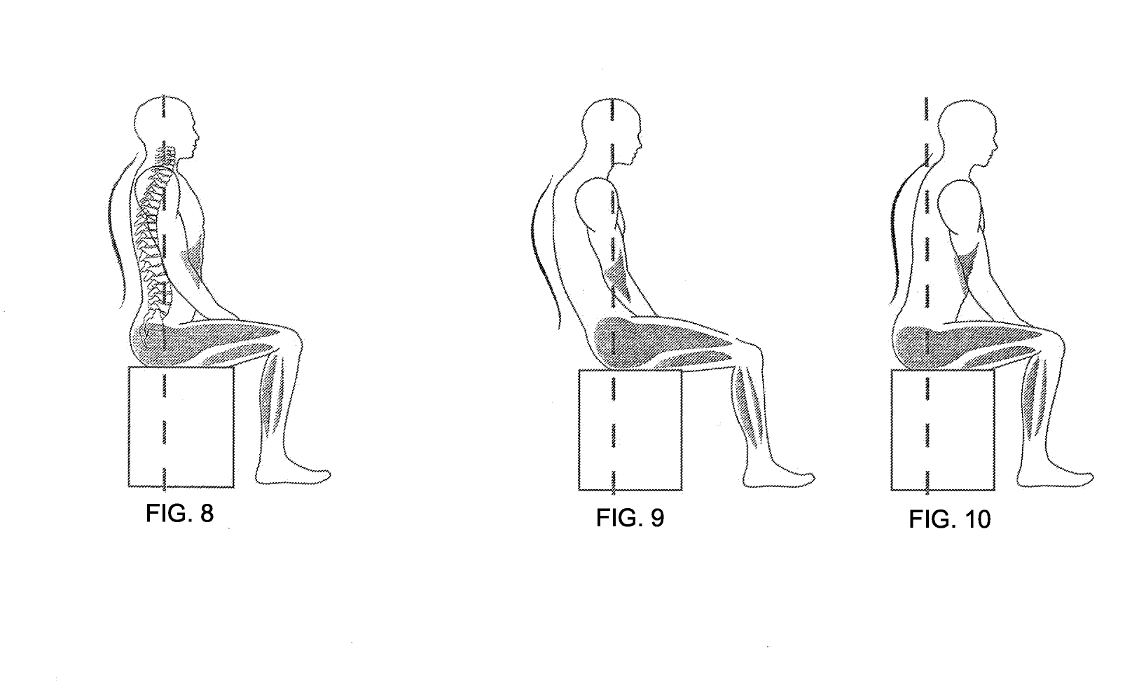

Referring now to FIG. 13, a person standing with incorrect posture primarily characterized by rounded shoulders, also has the head forward, chest leaning forward, back arched forward, and knees bent.

Referring now to FIG. 14, a person standing with incorrect posture with the back slouched, or arched, backwards, also has the knees bent.

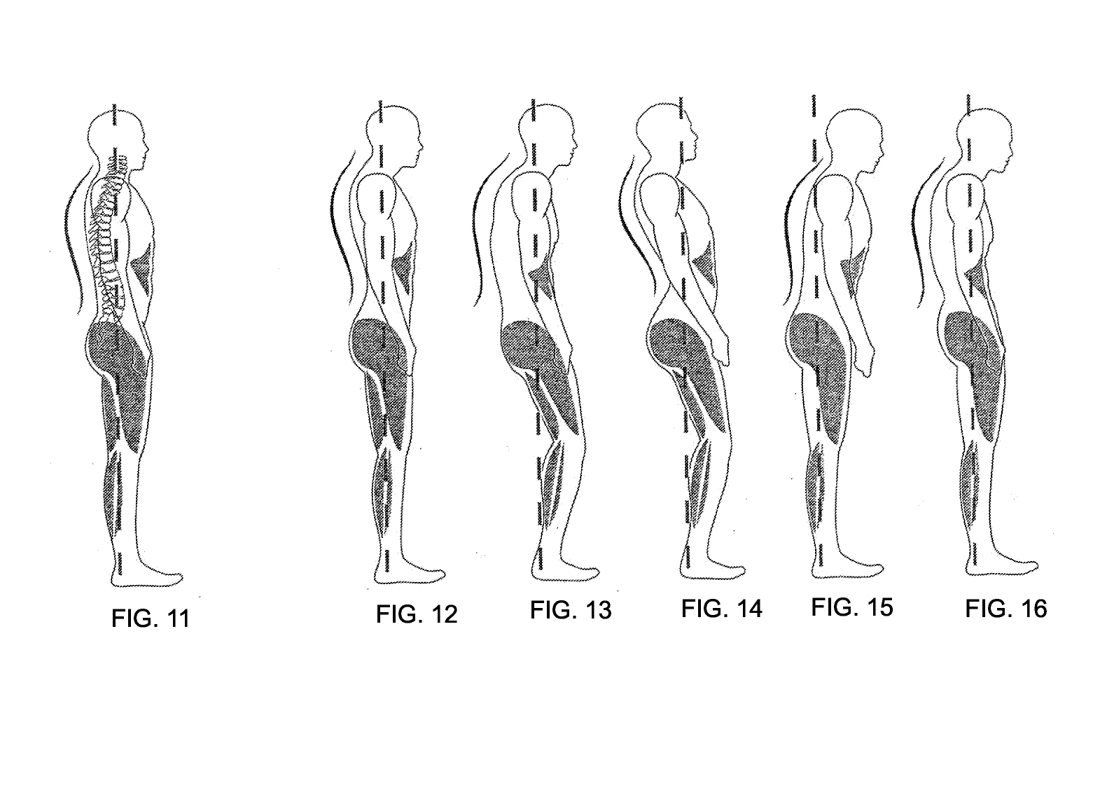

Referring now to FIG. 15, a person is shown standing with incorrect posture with a forward-leaning head.

Referring now to FIG. 16, a person is shown standing with incorrect, slumping posture, with the head forward, and the back slouched, or arched, forward.

Although only several embodiments of the present invention have been shown and described herein, it will be obvious to those having ordinary skill in the art that changes and modifications may be made thereto without departing from the scope and spirit of the invention as hereinafter claimed.

What is claimed is:

1. A posture training device comprising
a housing immovably attachable to a user's chest, said housing enclosing an electro-chemical power source, a micro-controller unit, a three-axis accelerometer, an electric motor coupled to a rotatable, out-of-balance weight, a plurality of light-emitting diodes, an ON/OFF switch, and a selector switch, all of which are powered by the electro-chemical power source;
wherein the device can be booted to an ON state by depressing said ON/OFF button, selectivity of the device and a desired posture position can be set by depressing said selector switch; and
wherein vibration negative feedback is provided whenever the user slumps or slouches outside limits of movement permitted by a chosen sensitivity level, and vibration positive feedback is provided when correct posture is reestablished.
